(19) United States
(12) Patent Application Publication
(10) Pub. No.: US 2024/0282404 A1
(43) Pub. Date: Aug. 22, 2024

(54) Protein engineering workflow using a generative model of protein families

(71) Applicant: NE47 Bio, Inc., Hillsborough, NC (US)

(72) Inventors: Tristan Bepler, Hillsborough, NC (US); Timothy F. Truong, JR., Flushing, NY (US)

(21) Appl. No.: 18/443,534

(22) Filed: Feb. 16, 2024

Related U.S. Application Data
(60) Provisional application No. 63/446,545, filed on Feb. 17, 2023.

(51) Int. Cl. G16B 15/00; G16B 40/20

(52) U.S. Cl. CPC G16B 15/00; G16B 40/20

(57) ABSTRACT

A retrieval-augmented framework leverages a generative protein language model of whole protein families. The model is configured and trained on homologous sequences and learns to generate sets of related proteins as sequences-of-sequences across very large numbers (e.g., tens of millions) of natural protein sequence clusters. In order to capture conditioning between sequences in an order independent manner (typically, the order of sequences within a family is arbitrary) and to generalize to large context lengths, the model leverages a transformer layer that models order-dependence between tokens within sequences and order-independence between sequences. Upon training, the model is used in protein engineering workflows, such as controllable design of protein sequences and variant effect prediction.

### CROSS-REFERENCE TO RELATED APPLICATIONS

This application claims priority to Ser. No. 63/446,545, filed Feb. 17, 2023, titled “A generative model of protein families as sequences-of-sequences.”

### BACKGROUND OF THE INVENTION

Proteins carry out most of the biological functions at the molecular level of life. The function of a protein is encoded by its specific amino acid sequence and the three-dimensional structure that the sequence folds into. Engineering proteins for novel and enhanced function is a key problem in pharmaceuticals and biotechnology and involves designing novel sequences or modifying existing natural proteins for these purposes. Deep mutational scans and directed evolution experiments have been used to successfully design novel proteins, but they can be costly and difficult to implement, which makes these experimental methods inapplicable for many proteins and functions of interest. Accurate computational models of sequence-function relationships can narrow down the protein sequence search space, reduce the need for expensive experiments, and enable the design of more novel proteins and functions.

Protein language models have emerged as promising methods for understanding and designing protein sequences. In particular, generative models offer a natural way to produce new protein designs. By training on large corpuses of natural protein sequences, these models learn evolutionary constraints on sequence space. They can then be used either to generate realistic sequences directly by sampling, or to identify promising protein sequence variants by predicting the relative fitness of the variants of interest using the sequence likelihoods as a proxy.

Traditionally, family-specific models learn evolutionary constraints specific to the protein family of interest by training on a multiple sequence alignment (MSA) of homologous sequences. However, this is ineffective for protein families with few sequences due to the lack of sufficient training data and inability to exploit information across families. These models also assume that MSAs are accurate, and they cannot model novel insertions or deletions (indels) not present in the training MSA.

Evolutionary sequence models are well established methods in biological sequences analysis. To model protein families, these models search large protein sequence databases for homologs, align the positions of these homologs in an MSA, and then fit statistical sequence models to the MSA. Common models include site independent models, profile HMMs, and coupling models. Newer variants incorporate higher order correlations between sequence positions by training a VAE or by building phylogenetic trees. These approaches are often referred to as “alignment-based” and must be fit on a family-by-family basis, requiring large numbers of members to generalize. A significant limitation of these models is that they assume the MSA is an accurate model of the evolutionary process generating the sequences, when in fact, MSA algorithms inevitably make alignment errors; regions with long insertions or lots of gaps can be particularly problematic.

Unconditional protein language models that do not condition on homologs at inference have emerged as powerful methods for understanding and generating protein sequences. Both bidirectional models and autoregressive generative models have demonstrated competitive performance for variant function prediction. The latter type of model has the advantage of being able to score indels, but both cannot integrate evolutionary context not present in the trained model parameters. In contrast to family-specific evolutionary sequence models trained on sequences derived from a specific protein family, these protein language models are pre-trained on large protein databases that span all known sequences. This enables them to learn evolutionary constraints that generalize across families to improve predictions for small families with few homologs, but they generally underperform family-specific models for larger families.

Hybrid models such as Tranception and TranceptEVE combine unconditional language models with family-specific models to enable specialization to protein families. Nonetheless, it is unclear how to use these models to generate sequences with novel indels, and predictions from the family-specific models do not directly benefit from transfer learning across families.

Conditional protein language models fit between the unconditional and family-specific paradigms. Only a few works have explored this direction to date. Masked language models of whole MSAs are able to integrate evolutionary context directly for conditioning site predictions, but they are unable to model insertions in the alignment. Ram and Bepler use an encoder-decoder framework to generate new sequences conditioned on an MSA, which removes the insertion limitation of Rao et al., but still requires conditioning on aligned input sequences. Notin et al. combine predictions from an unconditional language model and an alignment-based model and show that integrating retrieval-based methods with protein language models can improve variant function prediction performance. However, the reliance on an alignment-based model means that the combined model is still limited by the constraints of MSAs.

Retrieval-augmented language models have shown impressive results in natural language processing, especially on Question Answering (QA) tasks. These models incorporate a database search as part of the text generation process in order to generate new text conditioned on prototypes found in the database. In this way, they are conceptually similar to the conditional protein language models above. Retrieval-augmented approaches have the advantage of not requiring the entire training corpus to be encoded within the model parameters and the ability to easily integrate new data without retraining by simply adding it to the retrieval database.

### SUMMARY OF THE INVENTION

According to this disclosure, a retrieval-augmented framework leverages a generative protein language model of whole protein families. The approach frames the protein sequence generation problem as a sequence-of-sequences problem to incorporate retrieved-sequence conditioning, thereby providing a fundamentally different paradigm than that employed by current retrieval-augmented models in natural language processing. To this end, a generative protein language model, referred to herein as a Protein Evolutionary Transformer (PoET), is configured and trained on a large set of homologous sequences. By learning to generate sets of related proteins as sequences-of-sequences across very large numbers (e.g., tens of millions) of natural protein sequence clusters, PoET generalizes about evolutionary processes across protein families, and it avoids issues related to conditioning on MSAs. In order to capture conditioning between sequences in an order independent manner (typically, the order of sequences within a family is arbitrary) and to generalize to large context lengths, PoET leverages a transformer layer that models order-dependence between tokens within sequences and order-independence between sequences.

In a representative protein engineering workflow, PoET is configured as a generative model of whole protein families and used for controllable design of protein sequences and variant effect prediction. PoET generates sets of homologous proteins as sequences-of-sequences. In a typical use case, the model is controlled by providing it with a prompt, namely, a set of sequences that represent homologues, family members, or some other grouping of related sequences that represent a protein of interest, and receiving an output from the model. Given a prompt, the model enables various protein engineering workflows, e.g., scoring of arbitrary sequences to predict sequence fitness and rank variants, mapping of fitness of single substitution variants, identifying mutable hotspots and designing combinatorial variant libraries, generating bespoke, high order variants (by sampling from the model), and exploring diverse sequence space of a protein, and the like.

The foregoing has outlined some of the more pertinent features of the subject matter. These features should be construed to be merely illustrative. Many other beneficial results can be attained by applying the disclosed subject matter in a different manner or by modifying the subject matter as will be described.

### BRIEF DESCRIPTION OF DRAWINGS

FIG. 1 depicts a sequence of three protein sequences of different lengths;

FIG. 2 depicts a probability function of a sequence-of-sequences (such as depicted in FIG. 1) that is used by PoET to generate each token in the sequence;

FIG. 3 depicts an inter-sequence relative position encoding scheme used in sequence-of-sequences self-attention within PoET that is invariant to sequence ordering;

FIG. 4 depicts a representative PoET architecture according to this disclosure;

FIG. 5 depicts a first algorithm corresponding to the TieredTransformerEncoderLayer shown in FIG. 4;

FIG. 6 depicts a first model variant, referred to herein as Prefix-LM POET with encoder-decoder architecture, that is trained on a prefix language modeling objective;

FIG. 7 depicts a second algorithm corresponding to the TieredTransformerDecoderLayer shown in FIG. 6;

FIG. 8 depicts a second model variant, referred to herein as MLM PoET, that is trained on a Masked Language Modeling (MLM) objective, rather than a causal or prefix language modeling objectives;

FIG. 9 depicts a third model variant, referred to herein as Multi-task PoET, that provides for an encoder-decoder prefix-LM PoET additionally trained on an MLM objective;

FIG. 10 depicts a third algorithm, referred to herein as PoETEmbed, by which the POET model creates useful pre-residue embeddings;

FIG. 11 depicts a first protein engineering workflow, namely, for variant prioritization;

FIG. 12 depicts a second workflow, namely, sampling sequences from the model for generating new sequences that have a same function as a known set of sequences;

FIG. 13 depicts several examples of chaining natural language and protein sequences for prompt engineering for the task of using the model to specify a protein family using natural language descriptions;

FIG. 14 depicts a third workflow, namely, a workflow to facilitate sequence to function learning;

FIG. 15 depicts a fourth workflow, namely, generating a per-residue annotations model using PoET;

FIG. 16 depicts a fifth workflow, namely, generating a structure prediction model using PoET; and

FIGS. 17-20 depict various user interfaces for receiving information to generate prompts for use against the POET model in various use cases.

### DETAILED DESCRIPTION OF THE INVENTION

By way of background, the following terms have the following meaning.

A “language model” is a probabilistic model of sequences. In the case of natural language, language models typically describe the probability of sentences or documents. In the case of proteins, they model the probability of amino acid sequences. Being simply probabilistic models, language models can take on many specific incarnations, e.g., from column frequencies in multiple sequence alignments to Hidden Markov Models to Potts models to deep neural networks.

A “generative model” is a model of a data distribution, p(X), joint data distribution, p(X, Y), or conditional data distribution, p(X|Y=y). It is usually framed in contrast to discriminative models that model the probability of the target given an observation, p(Y|X=x). Here, X is observable, for example the protein sequence, and Y is a target that is not observed, for example the protein structure or function. Conditional generative and discriminative models are related by Bayes' theorem. Language models are generative models.

An “autoregressive language model” is a language model that factorizes the probability of a sequence into a product of conditional probabilities in which the probability of each token is conditioned on the preceding tokens, p(x₁ . . . x_{L})=Πᵢ₌₁^{L}p(xᵢ|x₁ . . . xᵢ₋₁). Examples of autoregressive language models include k-mer (also known as n-gram) models, Hidden Markov Models, and typical autoregressive recurrent neural network or generative transformer language models. These models are called autoregressive because they model the probability of one token after another in order.

### Protein Evolutionary Transformer (PoET)

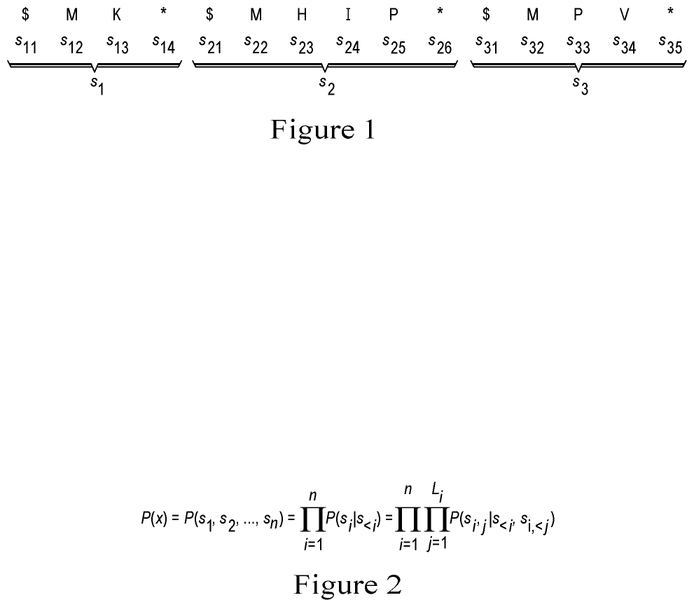

In one embodiment, PoET is an autoregressive generative model of the distribution over protein families, where each family is generated as a sequence-of-sequences. Specifically, it models the distribution P(X=x), where x=s₁, s₂, . . . , sₙ is the concatenation of n sequences sᵢ from the same family, and each sequence sᵢ=s_{i,1}, s_{i,2}, . . . , s_{i,Li} is a sequence of Lᵢ amino acids padded by a start and end token. For example, FIG. 1 depicts a sequence of three protein sequences of lengths 4, 6, and 5 with start token denoted by $ and stop token denoted by *.

When referring to a sequence-of-sequences, sᵢ, which has one index i, refers to a sequence of tokens, namely the ith sequence in the sequence-of-sequences, whereas s_{i,j}, which has two indices i,j, refers to one token, namely the jth token of the ith sequence. As used herein, x denotes the full sequence-of-sequences. Preferably, PoET generates each token in a sequence x one at a time, decomposing the probability of x as set forth in FIG. 2. The order of the individual sequences in a sequence-of-sequences is arbitrary, and PoET leverages a transformer-based architecture to exploit this order invariance, as is now described.

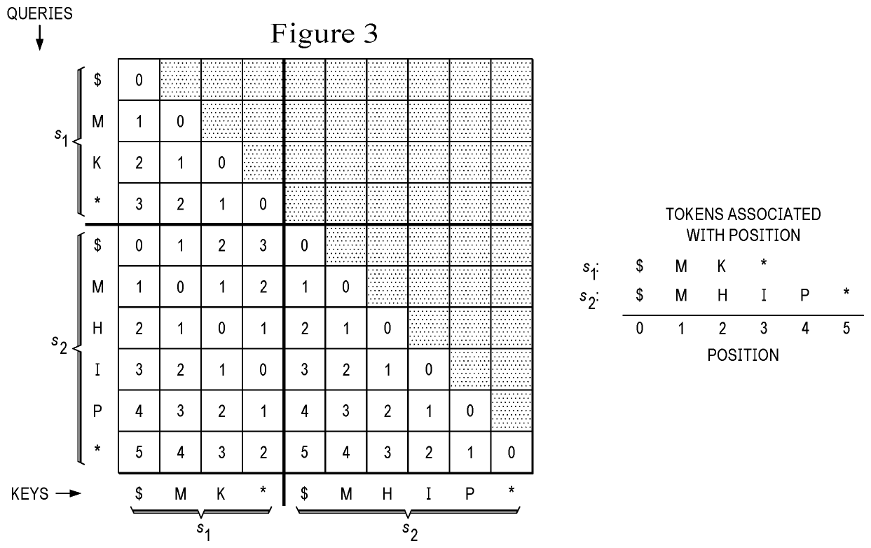

FIG. 3 depicts an inter-sequence relative position encoding scheme used to facilitate sequence-of-sequences self attention, as will be described. In this example, the first two sequences in the sequence-of-sequences (of FIG. 1) are shown. As depicted on the left side, the relative positions between tokens in the sequence pair are shown, and the right side depicts the resulting tokens associated with a set of absolute positions. The encoding scheme is invariant to sequence ordering, and further details of the scheme are referenced below.

The following provides additional details regarding the Protein Evolutionary Transformer (PoET).

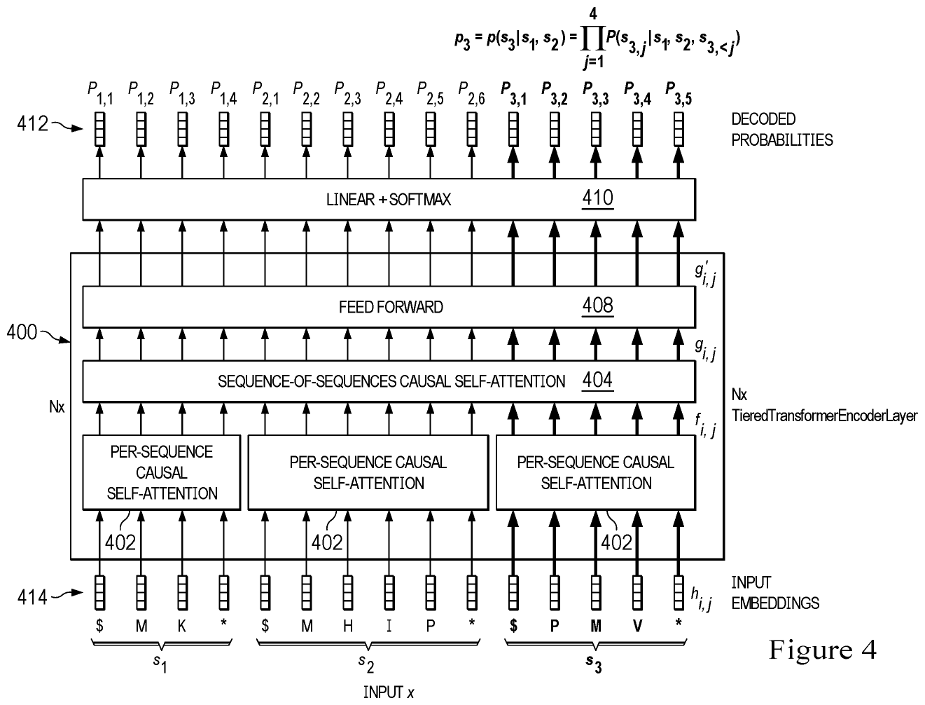

With reference now to FIG. 4, the transformer-based architecture of this disclosure comprises a specialized transformer encoder layer 400 to capture order invariance between sequences while preserving order-dependence between tokens within sequences. In this embodiment, this is accomplished using two attention modules: (i) a within-sequence module 402 in which the representation at each position of each sequence is updated based on attending only to the other tokens within this sequence, and (ii) a between-sequence module 404 in which the representation at each position of each sequence is updated based on attending to all sequences within the sequence-of-sequences. This tiered approach ensures capture of long-range dependencies between sequences and uniquely allows the model to extrapolate to much longer context lengths than used during training, thereby improving sequence generation and performance on downstream tasks. In a particular implementation, the PoET model is a stack of these layers with causal self-attention. As also depicted, outputs from the between-sequence module 404 are applied to a feed forward module 408, which prepares the outputs for further processing, with the result generated by the transformer encoder layer 400 then processed through a Linear+SoftMax module 410 to generate a set of decoded probabilities 412 corresponding to a set of input embeddings 414 received by the model. In particular, a linear layer in module 410 takes the decoded activations and projects them to a size of the vocabulary (as logits). The SoftMax layer in the module 410 takes these logits and generates next-token probabilities. For example, a next predicted token is the argmax of the softmax output.

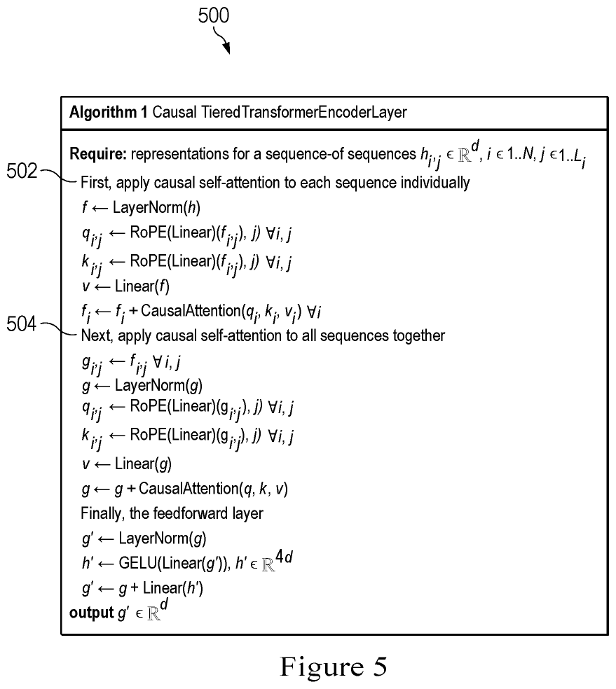

The following provides additional details regarding how the transformer encoder layer operates. Given an input sequence x=s_{i,j}, i∈1 . . . n, j∈1 . . . Lᵢ of amino acids and start/stop tokens, this sequence is first converted into a sequence of continuous embeddings h_{i,j} by mapping each token to its learned embedding:

$${h}_{i , j} = {W}_{si , j} , {s}_{i , j} ∈ AA ⋃ \{ START , STOP \} , {W}^{∈} {R}^{\vert {AA}_{⋃} \{ START , STOP \} \vert \times d}$$

where AA is the set of 20 standard amino acids, and W is a matrix of learnable embeddings of dimension d. Next, the embeddings h_{i,j} are transformed by N layers of a first algorithm (Algorithm 1), which is referred to herein as a Causal TieredTransformerEncoderLayer, and which is specified by the listing 500 in FIG. 5. As noted above, this transformer is a specialized layer for processing a sequence-of-sequences that is invariant to the order of the individual sequences, and that extrapolates to context lengths substantially longer than the training context length.

The TieredTransformerEncoderLayer 500 is composed of two phases. In the first phase, at 502, causal self-attention is applied independently to each sequence hᵢ of the input sequence-of-sequences, transforming them into new sequences ƒᵢ=PerSequenceSelfAttn(hᵢ). This is the operation of the within-sequence module 402 in FIG. 4. Preferably, relative positional information is encoded by applying Rotary Positional Encodings (RoPE) to the queries and keys before applying self-attention; the absolute position for ƒ_{i,j} is j.

The second phase, at 504, applies causal self-attention to the entire sequence-of-sequences by concatenating the individual ƒᵢ from the previous layer into one sequence before applying self-attention: g_{i,j}=SequenceOfSequencesSelfAttn([ƒ_{<i};ƒ_{i,<j}]). In order to make self-attention in this phase invariant to sequence order, the inter-sequence relative positional encoding scheme of FIG. 3 is used, namely: for g_{i,j} the absolute position is j. Just as in the first phase, the absolute position for tokens in the ith sequence gᵢ is independent of the position i of the sequence in the sequence-of sequences. Thus, the positional information encoded by RoPE in this layer alone does not distinguish between the positions of tokens in different sequences. For example, the relative position between the first token of the first sequence ƒ_{1,1} and the first token of the second sequence ƒ_{2,1} is 0. The fact that these two tokens come from two different sequences is encoded by the first phase, which operates on the two sequences independently. This inter-sequence relative positional encoding scheme has several useful properties: it encodes the fact that amino acids at similar absolute positions in homologous proteins are more likely to be drawn from the same distribution, and it limits the maximum relative position encoding needed to the number of tokens in an individual protein sequence, rather than the number of tokens in a sequence-of-sequences, allowing the model to generalize to longer sequences-of-sequences than seen during training.

Lastly, and referring back to FIG. 5, at 504, the output from the last TieredTransformerEncoderLayer, g_{i,j}, is decoded into token probabilities by applying a linear transformation P(s_{i,j})|s_{<i},s_{i,<j})=p_{i,j}(s_{i,j})=Linear(g_{i,j}). Here, p_{i,j} is a vector of probabilities, one for each distinct token ∈ AAU{START,STOP}, and p_{i,j}(s_{i,j}) is the probability of the token s_{i,j} according to p_{i,j}.

### Training

The POET model is trained on large sets of homologous sequences. In one example training, POET is trained on 29 million sets of related sequences, with each set corresponding to a sequence in UniRef50 Version 2103, and containing all its homologs in UniRef50 found using the Diamond search tool. Any sets with fewer than 10 homologs are removed. To avoid overfitting on promiscuous sequences which may belong to a large number of sets, each set is sampled with weight inversely proportional to the size of the set (“inverse count” sequence weighting).

To find homologs in UniRef50 using Diamond, an all-against-all search was carried out, and that search returns, for each sequence in UniRef50, a set containing all its putative homologs in UniRef50. Each such set is a “Diamond-UniRef50 Cluster.” Diamond was used over other homology search tools due to its high performance (>100× speed of BLAST).

To form a training example, sequences are then sampled without replacement from a “Diamond-UniRef50 Cluster” until the total number of tokens reached a predetermined limit, and then concatenated to form a sequence-of-sequences. Each sampled UniRef50 sequence is replaced with a UniRef100 sequence by sampling a random UniRef100 sequence from the same UniRef50 cluster as the UniRef50 sequence being replaced. The UniRef100 sequences are randomly sampled with weight inversely proportional to the size of the UniRef90 clusters to which they belong. Preferably, each UniRef100 sequence is sampled at most once in each sequence-of-sequences.

As a final data augmentation, preferably the order of the tokens in a sequence-of-sequences is reversed with probability 50% (i.e. all sequences in a sequence-of-sequences are ordered from either N-terminus to C-terminus only or C-terminus to N-terminus only). This augmentation has been shown to improve the performance of other protein language models.

Following this sampling procedure, the order of sequences in a sequence-of-sequences is random, which promotes order invariance.

### Loss Function

In the above-described example, the model is trained to minimize the negative log likelihood of the next token:

$$- log P ( x ) = - {\sum }_{i = 1}^{n} {\sum }_{j = 1}^{{L}_{i}} log P ( {s}_{i , j} \vert {s}_{i , < j} )$$

Preferably, the model is also trained to predict the next token with the order of all tokens (within a single protein sequence) reversed and during inference, compute model likelihoods of sequences in their original order and in reverse order.

### Variant Models

The loss function described above is known as a causal language modeling loss function because the probability of the next token is predicted based on only past tokens, without looking at any future tokens. This loss function is particularly useful for sequence generation tasks because it decomposes the probability of a sequence such that it can be generated one token at a time from beginning to end. When, however, the model is not being asked to generate a sequence this causal restriction is needlessly restricting. For example, when prompting the model by providing it a sequence of sequences related to the sequence that is desired to be generated, the representation of the first sequence is independent of the second sequence, even though information about the second sequence may be helpful for building a better representation of the first sequence. A prefix language modeling (Prefix-LM) objective solves this issue by allowing the model to attend fully to a prefix of a sequence, in this case, the part of the sequence that corresponds to the prompt.

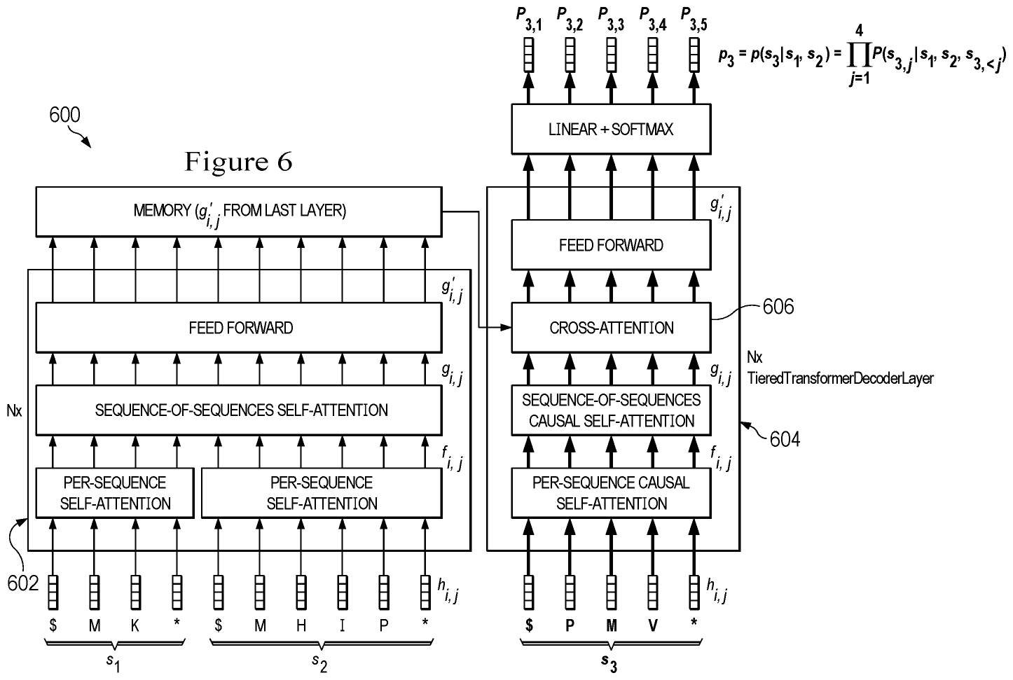

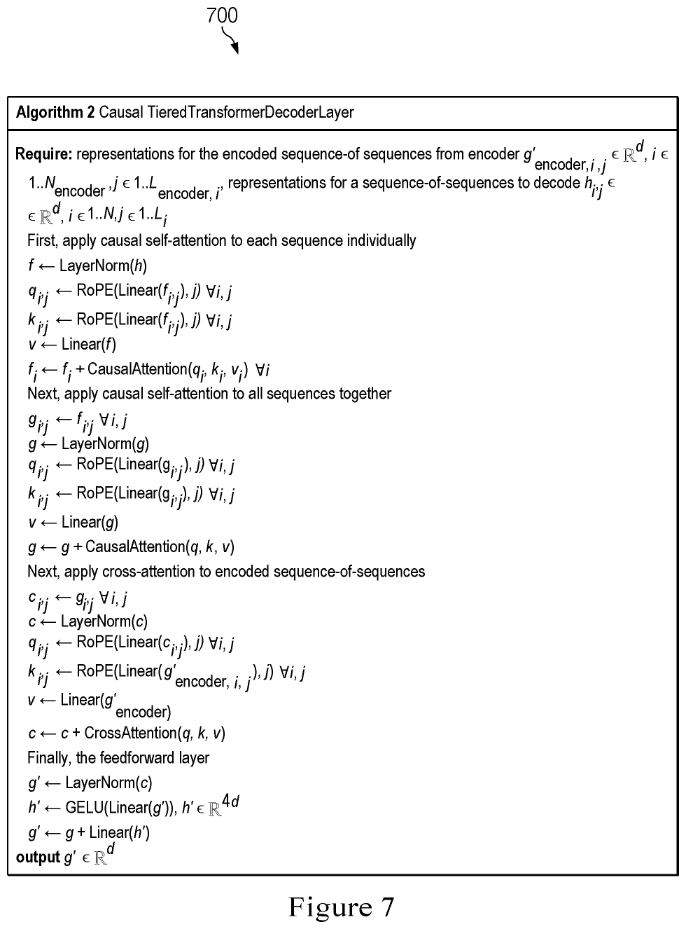

Thus, and with reference now to FIG. 6, a first variant model, which is referred to herein as Prefix-LM PoET, is depicted as reference numeral 600. In this example (with the sequence-of-sequences), the first two sequences are processed in an encoder portion 602 of the transformer, and the next sequence is process in a decoder portion 604. As can be seen, the decoder portion 606 also includes an additional cross-attention function 606 that receives the output of the encoder. The cross-attention function 606 applies cross-attention to encoded sequence-of-sequences. More specifically, the Prefix-LM POET variant model implements a second algorithm (Algorithm 2) that is depicted in the listing shown in FIG. 7. This algorithm is referred to as TieredTransformerDecoderLayer 700 due to the inclusion of the decoder portion 604, and to differentiate from the TieredTransformerEncoderLayer 500 that was depicted in FIG. 5 (for the regular PoET model).

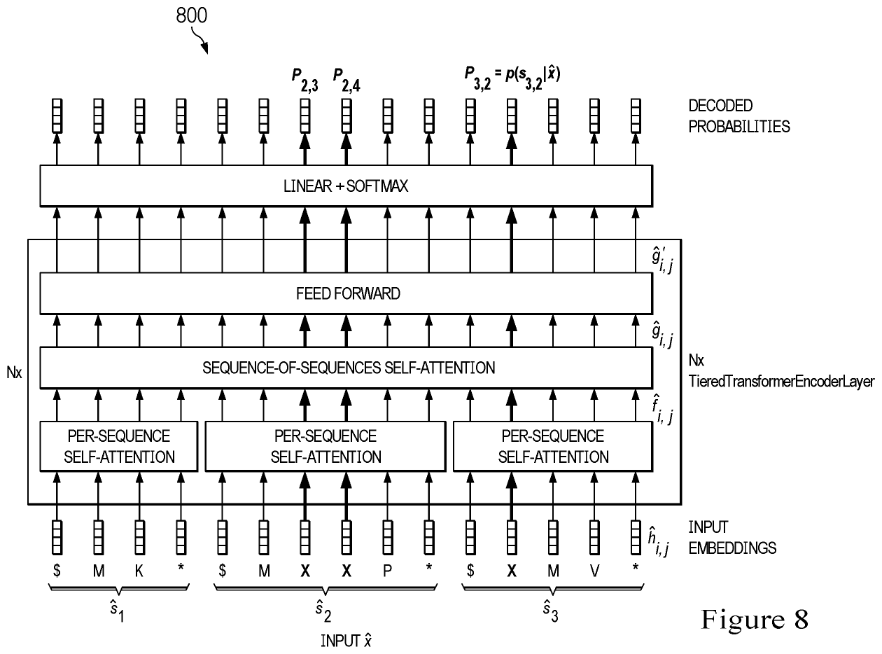

Referring now to FIG. 8, a second variant model, which is referred to herein as MLM PoET, is depicted as reference numeral 800. MLM POET is a variation of the PoET model (of FIG. 4) and that is trained on the masked language modeling (MLM) objective, rather than the causal or prefix language modeling objectives for causal and prefix-LM PoET. The MLM objective replaces a subset of the tokens in the input sequence with either a special mask token or a random token from the vocabulary, and then asks the model to predict the replaced token. In the context of PoET, the corresponding loss function is:

$$- log P ( x , \hat{x} , m ) = - {\sum }_{i = 1}^{n} {\sum }_{j = 1}^{{L}_{i}} {𝕝}_{{m}_{i , j}} log P ( {s}_{i , j} \vert \hat{x} )$$

In this function, x\hat{ }=s\hat{ }₁, s\hat{ }₂, . . . , s\hat{ }ₙ is the masked version of x, and m_{i,j} are random variables indicating whether or not the jth token of the ith sequence is masked. In one example embodiment, 15% of tokens are masked, and among masked tokens, 90% of tokens are replaced with mask tokens, and 10% are replaced by random tokens. These proportions can be chosen differently and even varied during training.

The MLM objective as implemented in FIG. 8 is useful because it allows the model to attend to all tokens in each sequence, rather than only causal attention (for the full sequence in causal language modeling and for the suffix in prefix language modeling).

The Causal PoET architecture in FIG. 4 can adapted to perform MLM by simply replacing “Causal Self-Attention” in (modules 402 and 404) with “Self-Attention.”

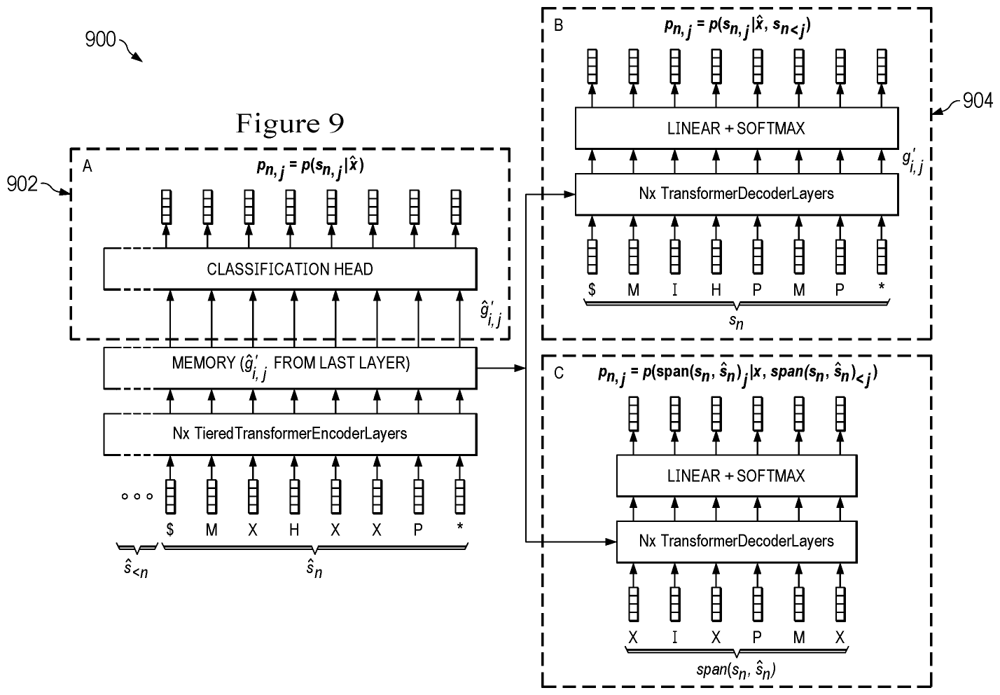

The encoder-decoder variant of Prefix-LM POET depicted in FIG. 6 (and described by Algorithm 2 in FIG. 7) can be enhanced to perform masked language modeling in addition to prefix language modeling by adding an additional decoder that is conditioned on the memory outputted by the encoder portion and training it to optimize the masked language modeling objective. This variant model is depicted in FIG. 9 as reference numeral 900. This model is referred to herein as Multi-task PoET: Encoder-Decoder Prefix-LM POET with MLM-like objectives. As depicted in FIG. 9, there are several ways to accomplish this, e.g., option A, which involves adding a classification head 902 to predict the original identities of masked tokens and train the model to optimize the MLM PoET model loss function described above. Option B involves adding a regular Transformer decoder 904 that autoregressively decodes each individual sequence in the sequence-of-sequences by performing cross-attention with the subsequence of the encoder memory that corresponds to the sequence being decoded. For option B, the loss function is similar to the MLM PoET model loss function, except that the model is allowed to condition on the true values of all previous tokens regardless of whether or not they are masked in the encoder. Thus, for Option B, the loss function is:

$$- log P ( x , \hat{x} , m ) = - {\sum }_{i = 1}^{n} {\sum }_{j = 1}^{{L}_{i}} log P ( {s}_{i , j} \vert \hat{x} , {s}_{i , < j} )$$

Referring back to FIG. 9, a third option, Option C, is the same as Option B except that in the decoded sequence, all consecutive unmasked tokens are replaced with a single mask token. For example, suppose that the original sequence is s=$MIHPMP*, and the masked sequence is s\hat{ }=$MXHXXP* (masked tokens are denoted by X). Then, and in Option C, the sequence to decode is given by a “span function” span(s,s\hat{ })=XIXPMX. The corresponding loss function is:

$$- log P ( x , \hat{x} , m ) = - {\sum }_{i = 1}^{n} {\sum }_{j = 1}^{L ( span ( {s}_{i} , {\hat{s}}_{i} ) )} log P ( span {( {s}_{i} , {\hat{s}}_{i} )}_{j} \vert \hat{x} , span {( {s}_{i} , {\hat{s}}_{i} )}_{< j} )$$

In the above loss function, the function L(·) gives the length of the input sequence.

The following describes obtaining homology augmented per-residue embeddings from PoET models. An embedding of a residue in a protein sequence is a real valued vector representing the unique context of that residue in its protein sequence. The corresponding embedding function is the function that transforms the residue into the embedding. Such an embedding function is useful when it places residues of similar contexts close to each other, and residues of differing contexts far away from each other (e.g. as measured by Euclidean distance). For example, two residues in two different protein sequences that both participate in the catalysis of the same chemical reaction may be considered similar, while a residue that does not participate in any catalysis may be considered dissimilar. The specific definition of the “context” of a residue is application dependent; the ideal embedding function works for any such definition of the context, or is able to adjust the embedding based on the definition of the context.

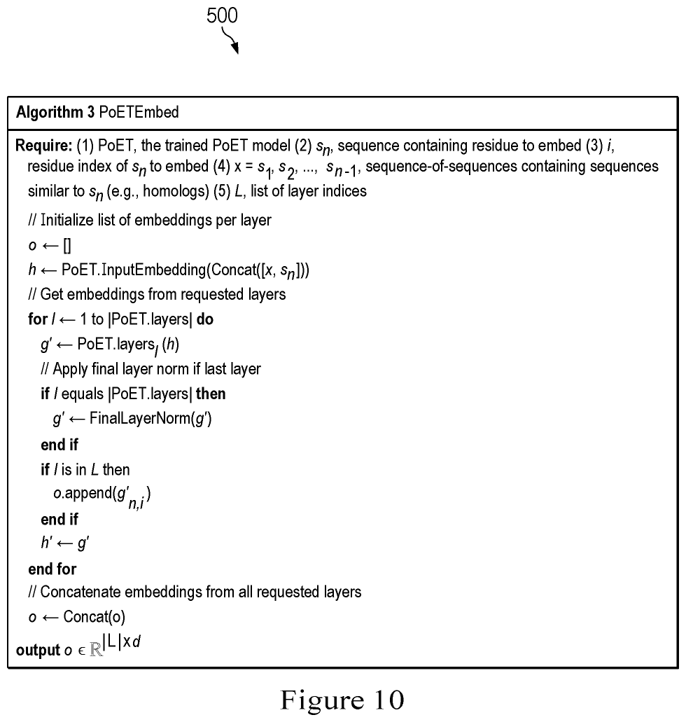

PoET models naturally create useful per-residue embeddings. In particular, these models include an embedding function that maps residues to a corresponding outputs g′_{i,j} of the “TieredTransformerEncoderLayer” (see, e.g., Algorithm 1) within the PoET models. These outputs are labeled as either g′_{i,j} of ĝ′_{i,j} in the POET architecture diagrams (FIGS. 4, 6 and 8-9). FIG. 10 depicts a third algorithm (Algorithm 3), referred to herein as PoETEmbed, for encoder-only PoET models. Embeddings can also be obtained from the decoder of (1) the encoder-decoder variant of Prefix-LM POET and (2) variants A and B of Multi-task PoET by using the output of the “TieredTransformerDecoderLayer” or TransformerDecoderLayer” instead (Algorithm 2, FIG. 6). The decoder of Option C of the Multi-task POET is not useful for this purpose.

The output of PoETEmbed (Algorithm 3) can be adjusted for different contexts by changing the sequence-of-sequences x, also called the “prompt”, that POET is conditioned on. Examples of different prompting methods are set forth below. Because the prompt generally contains protein sequences homologous to the protein family of interest, the embeddings obtained from PoET models are conveniently described as “homology augmented.” This homology augmentation differentiates PoET from other existing protein language models, which do not directly condition on homologous sequences and whose embeddings cannot be adjusted for different contexts.

### Applications and Use Cases

The POET models described herein facilitate various protein engineering workflows. Several example workflows are now described.

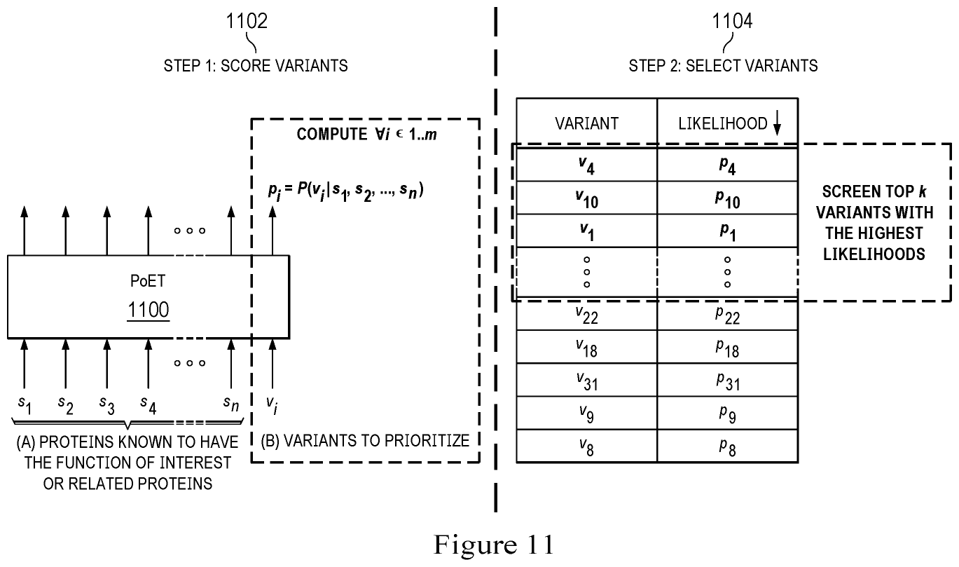

A first application/use case is variant prioritization, which is depicted in FIG. 11. Variant prioritization is applicable to all PoET variants, except MLM PoET. For Multi-task PoET variants, the decoder trained with the Prefix-LM objective is used. Protein variant fitness prediction is the task of assigning a score to each sequence in a set of variants {v₁, v₂, . . . , vₙ} of a target sequence t that accurately reflects the relative fitness of the variants. A protein variant vᵢ can be any sequence with a limited number of substitutions, insertions, and/or deletions relative to the target t that the experimenter believes may have improved fitness. Fitness refers to the value of any property of a protein sequence related to function that the experimenter is interested in optimizing e.g. thermostability, enzymatic activity, etc. As depicted in FIG. 11, the variant prioritization workflow with respect to the model 1100 involves a first step 1102 to score variants, and a second step 1104 to select the variants based on the scoring. In one embodiment of this workflow, fitness prediction with PoET works as follows.

In this embodiment, PoET predicts the fitness of a variant as the conditioned log-likelihood of the variant vᵢ given a set of sequences S homologous to the target t:

$${\hat{F}}_{i} ( S = \{ {s}_{1} , … , {s}_{m} \} ) = log P ( {υ}_{i} \vert {s}_{1} , {s}_{2} , … , {s}_{m} ) = {\sum }_{j = 1}^{{L}_{i}} log P ( {υ}_{i , j} \vert {s}_{1} , {s}_{2} , … , {s}_{m} , {υ}_{i , < j} )$$

In this example, the set S is retrieved by searching a large database of proteins such as UniRef100 for sequences homologous to t. The ColabFold protocol is used for retrieval, but this is not a limitation. The homologs form a diverse set of sequences that define the protein family of interest. By conditioning on these sequences, PoET infers evolutionary constraints on the protein family to improve fitness prediction. The sequences are conditioned on in an arbitrary order. In this example, the homologous sequences were subsampled and filtered to a reasonable context length for efficient inference, and conditional log-likelihoods are computed from different ensembles of homologous sequences. The final fitness prediction scores are obtained by averaging the conditional log-likelihoods across subsamples of the full set of retrieved homologous sequences:

$${\hat{F}}_{ensemble , i} ( S ) = \frac{1}{{N}_{ensemble}} {\sum }_{j = 1}^{{N}_{ensemble}} {\hat{F}}_{i} ( {S}_{j} ⊂ S )$$

Using the above-described technique, PoET has been shown to provide state-of-the art performance on variant fitness prediction. This performance demonstrates that PoET assigns higher likelihood to regions of sequence space with high fitness variants.

Accordingly, and as another example protein engineering workflow, POET is used to directly generate high fitness variants belonging to a protein family and, in particular, by conditioning PoET on sequences from the protein family and sampling from the resulting conditional distribution. Amino acids are sampled using various techniques, such as top k sampling, nucleus sampling, or beam search. Direct generation of variants makes the exploration of higher order mutants computationally tractable as sequence space grows exponentially large with number of mutations, making it impossible to explore this space by scoring all such variants. This application/use case (direct generation of novel protein sequences with user specified functions) is applicable to all PoET variants except MLM PoET. For Multi-task PoET variants, the decoder trained with the Prefix-LM objective is used.

Further details of using PoET to facilitate function-specific variant fitness prediction and sequence generation via prompt engineering are now described. As noted above, a PoET model as has been described has the ability to predict the “general” fitness of a protein variant, where “general” fitness can refer to any property that is related to the function of the protein. In practice, it is also desired to optimize specific properties of a protein. Because many properties of interest are correlated and together contribute to the general fitness of a protein (e.g. a more thermostable variant is also more likely to have higher expression or enzyme activity), optimizing general fitness is likely to optimize the true properties of interest, albeit indirectly. As noted above, PoET is useful for protein engineering because it is able to successfully predict general fitness by conditioning on and inferring evolutionary constraints from a diverse set of homologs of the target protein. By extension, and according to a further aspect, PoET is used to optimize specific properties of interest, preferably by conditioning on only the subset of relevant homologs that are known to or are predicted to display the specific properties of interest.

To this end, and based on experimental results, POET is able to learn function-specific evolutionary constraints for the target protein and property of interest, e.g., in the chorismate mutase indels dataset from ProteinGym. The dataset contains measurements of the catalytic activity, in E. coli, of 1130 natural chorismate mutase sequences, and 1618 designed chorismate mutase variants. The natural sequences are comprised of the target protein, a chorismate mutase found in E. coli, and homologs of the target protein found using the PSI-BLAST program for sequence search. The designed variants were selected by Monte Carlo sampling from a Potts model trained on an MSA of the natural sequences. This data presents an ideal scenario for selecting the subset of most relevant homologs; the subset of natural homologs that are measured to be functional are the ones selected. In the absence of such data, one could instead use predictions from another model, or other relevant known attributes of the sequences e.g. to optimize for activity at high temperatures, select only the homologs from thermophiles.

On the chorismate mutase dataset, it was found that the catalytic activity of designed chorismate mutase variants is better predicted when POET is conditioned on only the subset of functional natural sequences rather than all natural sequences (Δρ=0.2). In fact, PoET conditioned on functional natural sequences outperforms fully supervised methods, including a Gaussian process trained on mean embeddings from a BERT-like protein masked language model (Δρ=0.06). Such embeddings have been shown to be highly predictive of a variety of protein properties and provide a strong baseline. These fully supervised methods are trained on more data than PoET because they train on the measured catalytic activities of all the natural sequences, whereas PoET is simply conditioned on positively labeled natural sequences and does not have access to the measured activities. This enables PoET to be used with assays that only measure binary endpoints rather than continuous values.

Based on these results, PoET thus is shown to be useful for function-specific sequence generation. In the above scenario, and conditioned on the functional natural sequences, PoET was then to generate 1000 novel putative chorismate mutases using nucleus sampling with p=0.9.

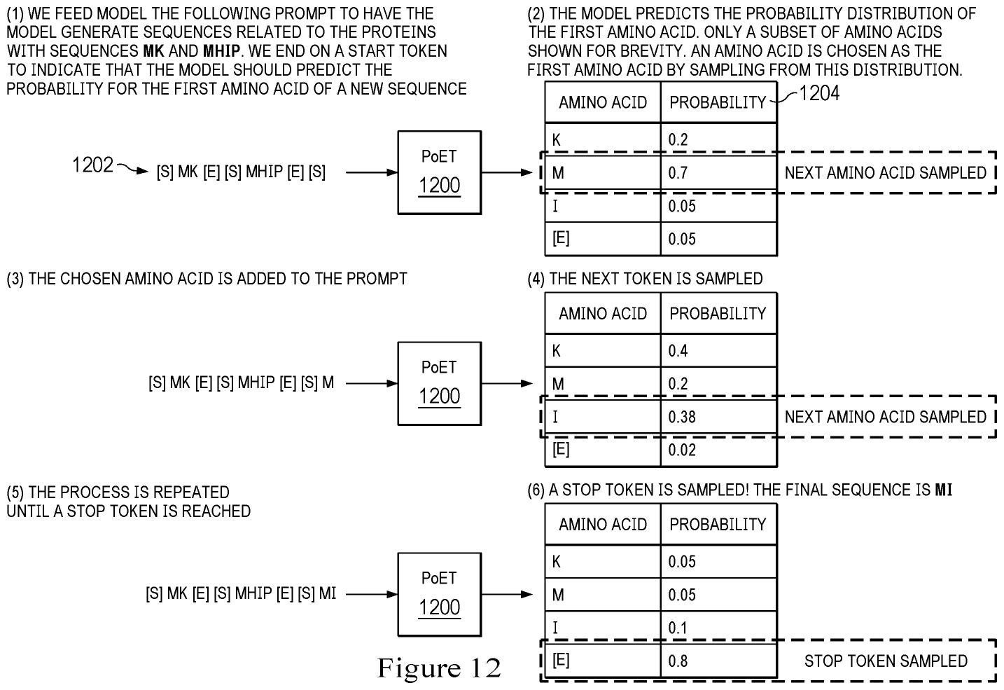

Generalizing, and with reference to FIG. 12, a POET model can be used to generate new sequences that are related to (i.e., has the same function as) a known set of sequences. This is accomplished by prompting the POET model with the known set of sequences, and then sampling from the model by using the predicted next token probabilities to determine the sequence of amino acids one at a time. Amino acids are sampled using various techniques, such as top k sampling, nucleus sampling, or beam search. FIG. 12 depicts the process for the POET model 1200. In this example, it is desired to have the model generate the sequences related to the proteins with sequences MI and MHIP. The prompt 1202 ends on a start token to indicate that the model should predict the probability for the first amino acid of a new sequence. As depicted, and based on the prompt 1202, the model has predicted the probability distribution 1204 of the first amino acids. Only a subset of amino acids in the distribution are shown for brevity. At step (2), an amino acid then chosen as the first amino acid by sampling from this distribution. Processing then continues at step (3) by adding the chosen amino acid to the prompt and re-applying the prompt to the model. At step (4), the next token is sampled. At step (5), the above-described process is repeated until at step (6) a stop token is reached. This completes the sequence generation process for the prompt.

In the above example, the kind of proteins that POET is prompted to generate is based on showing PoET examples of proteins that exhibit the function of interest, without directly specifying exactly what the function is. According to a further aspect of this disclosure, PoET can be extended to allow the function to be specified by natural language. In this embodiment, this is accomplished by extending the vocabulary to include tokens from natural language, and then prefixing protein sequences with a natural language description of the protein. The following example illustrates this approach.

In this example, the prompt begins with a natural language description of a protein, denoted by a special start token [SN], followed by the sequence of the protein described, denoted by the regular start token [S]. Next, the POET model is provided with the natural language description of the protein that it is desired for the model to generate, and which is similar to but not exactly the same as the first. Finally, the prompt ends with the regular start token to indicate that the model should begin generating a new sequence:

Prompt:
[SN] A PETase from a thermophillic organism. [E][S]LVDKYYET[E][SN]A
PETase from a mesophillic organism. [E][S]

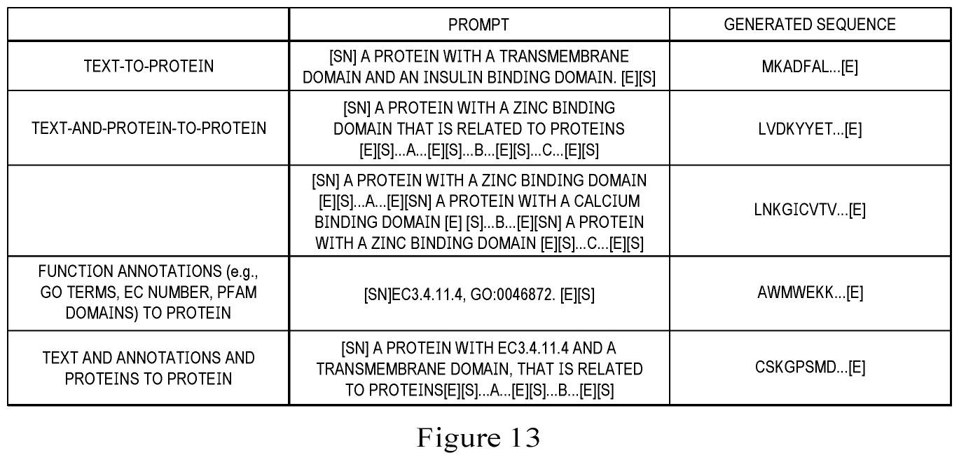

The generated sequence from this prompt is AWMWEKK[E]. Additional examples of this natural language-supported prompting are shown in FIG. 13. By chaining natural language and protein sequences in this way, the model is informed of what properties that the input sequences are known to have, and what properties the sequences that are desired to be generated have, and which may overlap only partially with the properties of the other properties. In the context of the POET model, each sequence composed of a natural language description and a protein sequence should be considered one sequence in a sequence-of-sequences, and such natural language descriptions can be prefixed to sequences corresponding to both free sequence generation and sequence infilling tasks, which are now described.

The following provides additional details regarding direct generation of novel protein sequences with user specified functions. In one embodiment, the protein family is specified using functional measurements. In particular, functional measurements of properties of interest can be included with each protein sequence (or some of them) in a prompt. This allows the model to learn the relationship between protein sequences and properties of interest, and allows the user to generate sequences with specific property values. Functional measurements can be omitted if they do not exist. For example, the following example prompt specifies the measured “Activity” before each sequence, and requests that the model generate a sequence with “Activity” equal to 5:

[SN]Activity:-1[E][S]DYET[E][SN]Activity:N/A[E][S]LYYEA[E][SN]Activity:5[E][S]

In another embodiment, the protein family is specified using structure. To condition on structure, the structure of each protein sequence can be included in the prompt, e.g., as 3D structure coordinates, or embeddings from a 3D structure embedding model such as an inverse folding model. An example of this type of prompt is as follows:

[SN]<structure coordinates or embeddings of following sequence>[E][S]DYET[E]
[SN]N/A[E][S]LYYEA[E]
[SN]<structure coordinates or embeddings of requested sequence>[E][S]

In another variant embodiment, the protein family is specified with multiple modalities (e.g., natural language, functional measurements, structure, etc.) by simply providing the data from each modality of interest before the protein sequence in the prompt.

Sequence infilling is the task of designing the amino acid sequence for only a part of a protein, while keeping other parts of the protein constant. This is a straightforward way to preserve protein function in the constant regions with high likelihood. Example applications include antibody CDR design and domain linker design.

To adapt PoET to perform sequence infilling, a prompt is modified as follows. A new start token [SI] is introduced to denote the infilling task. A sequence with the regions to infill masked out by a masking token are then specified. With this modified prompt, the model is then used to generate the complete sequence. In the example below, the model is prompted to infill the two regions of the sequence MK_TA_T denoted by the masking token_. After the normal start token [S], the model generates the infilled sequence, which replaces the masking tokens with sequences of amino acids. The infilled amino acids set off by asterisks but are not different from the normal amino acids:

Prompt:
[SI]MK_TA_T[E][S]

The generated sequence for this prompt is MK*SRA*TA*HK*T. In the POET context, each such sequence composed of a masked and an unmasked sequence should be considered one sequence in a sequence of sequences. PoET can be trained to perform both sequence infilling and free sequence generation simultaneously by simply training on sequences of sequences that contain both the free generation sequences, denoted by the regular start token [S], and the sequence infilling sequences, denoted by the start token [SI].

Still another application/use case for the POET architecture is Supervised Learning with homology augmented embeddings. In particular, the following subsections describe how homology augmented embeddings obtained from PoET via Algorithm 3 in FIG. 10 are used to develop improved supervised learning models that address other challenges in protein engineering.

Sequence to Function Learning. The aim of sequence to function learning is to create a mathematical model that predicts the ability of a protein to carry out its functions by learning from an existing dataset mapping protein sequences to quantitative measurements of those functions. Such predictive models can be used in black-box optimization algorithms to propose new hypotheses for protein sequences with enhanced function that can be validated in the lab. Per-residue POET embeddings can be used to create high quality sequence to function models by fitting machine learning models to predict measurements of function from the per-residue PoET embeddings of protein sequences. According to this aspect, each protein sequence is mapped to a sequence of per-residue PoET embeddings, one for each residue of the protein sequence. Then, each sequence of per-residue embeddings is reduced to a fixed length vector. Examples of such reduction functions include mean pooling, taking the embedding of the last residue of the protein sequence only, and computing a singular value decomposition (SVD). Finally, any supervised machine learning algorithm can be used to learn a function ƒ mapping the reduced embeddings to measurements of functions y\hat{ }ᵢ

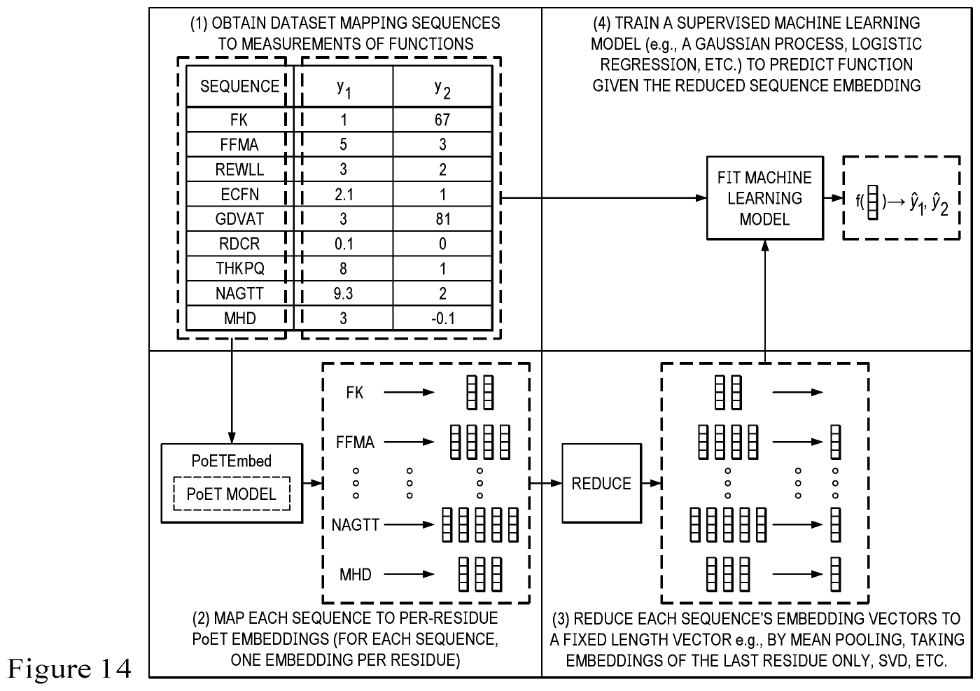

This process is illustrated in FIG. 14. As depicted, in step (1) a dataset mapping sequences to measurements of functions is obtained. At step (2), each sequence is then mapped to per-residue PoET embeddings. At step (3), each sequence's embedding vectors are reduced to a fixed length vector, e.g., by mean pooling, taking embeddings of the last residue only, SVD, or the like. At step (4), and given the reduced sequence embedding, a supervised machine learning model (e.g., a Gaussian process, logistic regression, or the like) is trained to predict the function. If the reduction in Step 2 and the machine learning model learned in Step 3 are differentiable with respect to their inputs, then the entire process (Steps 1-3) as depicted can be learned end-to-end, meaning that the parameters of the POET model used to create the per-residue embeddings can be finetuned simultaneously via backpropagation.

Per-Residue Sequence Annotation. Per-residue sequence annotation is the task of annotating a set of properties for each residue in a protein sequence. Examples of such properties include secondary structure, transmembrane, torsion angles, disorder, and binding sites. PoET models can be applied to per-residue sequence annotation by adding classifier and/or regression head(s) on top of per-residue PoET embeddings, and then finetuning the resulting model on a dataset containing annotations of the properties of interest. A “classification and/or regression head” refers to a neural network that takes as input the outputs of another neural network (the outputs could be the final outputs of the neural network, and/or internal ‘hidden’ states), such as a pretrained language model, and output predictions to classification and/or regression tasks. Classification and/or regression heads can have multiple outputs, and each output is a prediction for either a classification or regression task, and the outputs together can cover both classification and regression tasks simultaneously. Any existing finetuning technique can be used. “Finetuning” as applied to a neural network means further training (i.e., adjusting the parameters) of a neural network that has already been train on some task, and finetuning may be carried out on new task(s) with different loss function(s).

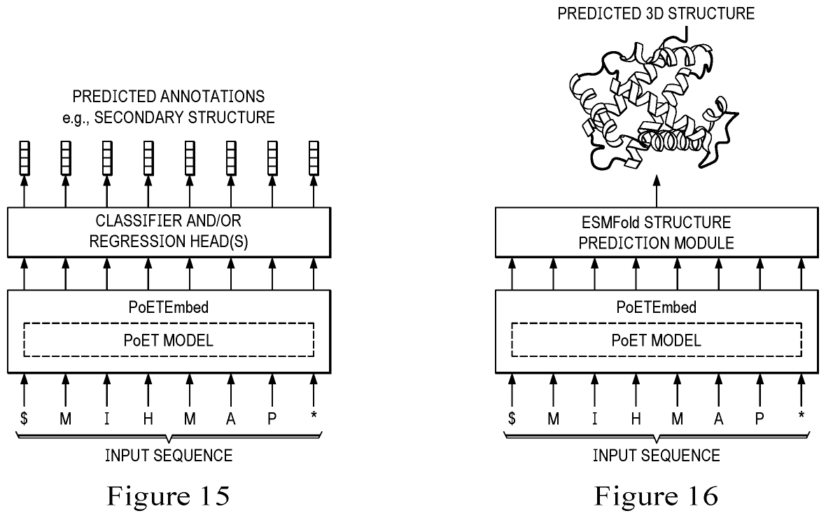

FIG. 15 illustrates a per-residue sequence annotation model utilizing a PoET model.

Another use case for POET is the task of predicting the 3D structure of a protein sequence. In known techniques, ESMFold currently provides a deep learning-based method to predict protein structure from its amino acid sequence. ESMFold uses a large protein language model for this purpose In this embodiment, PoET models are used for 3D structure prediction by replacing ESM2 per-residue embeddings (used in existing ESMFold approaches) in the ESMFold model with PoET per-residue embeddings, and training the resulting model using the same strategy as used in ESMFold. FIG. 16 illustrates such a model.

Summarizing, PoET is a Transformer-based autoregressive generative model of whole protein families. By framing family generation as a sequence-of-sequences generation problem, we are able to train across tens of millions of protein sequence clusters to encode fundamental rules of protein evolution into the POET model. This enables the model to generalize to protein families unseen during training and extrapolate from small numbers of conditioned-upon sequences. The sequence-of-sequences generative framework allows PoET to be used as a retrieval-augmented language model, generating new sequences conditioned on a set of sequences representing the family or other properties of interest. PoET has also been demonstrated to improve over other protein language models and evolutionary sequence models for variant fitness prediction across a wide range of deep mutational scanning datasets. PoET also enables efficient sequence generation and the generative distribution can be controlled via conditioning. Phage lysozyme- and chorismate mutase-like sequences sampled from PoET are novel and predicted to fold with high confidence. PoET can be backed by other sequence databases and naturally improves as databases grow without the need for retraining.

The techniques herein have significant advantages. As has been described, PoET is implemented as a retrieval-augmented protein language model by conditioning the model on sequences from any family of interest. This allows PoET to be used with any sequence database and to incorporate new sequence information without retraining. PoET is a fully autoregressive generative model, able to generate and score novel indels in addition to substitutions, and it does not depend on MSAs of an input family, removing problems caused by long insertions, gappy regions, and alignment errors. By learning across protein families, PoET extrapolates from short context lengths, thereby allowing it to generalize well even for small protein families. In addition, PoET can be sampled from and used to calculate the likelihood of any sequence efficiently.

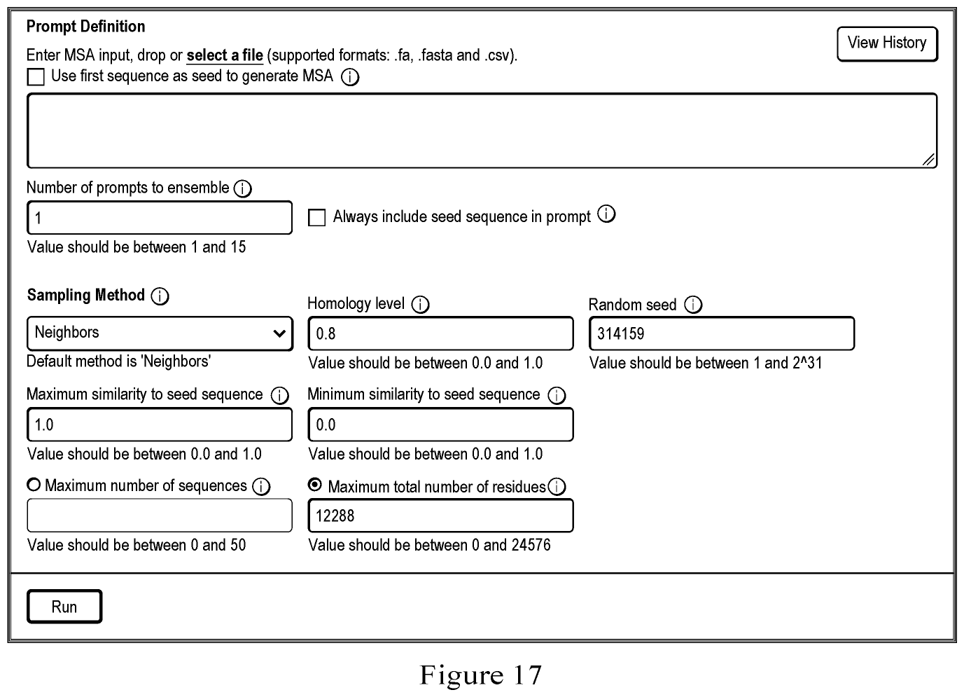

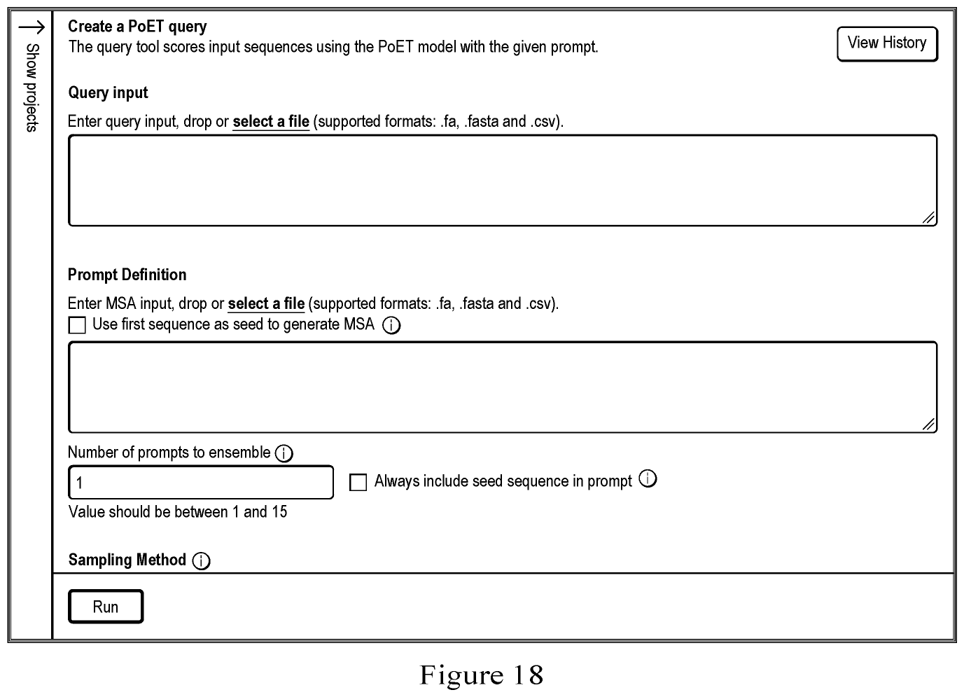

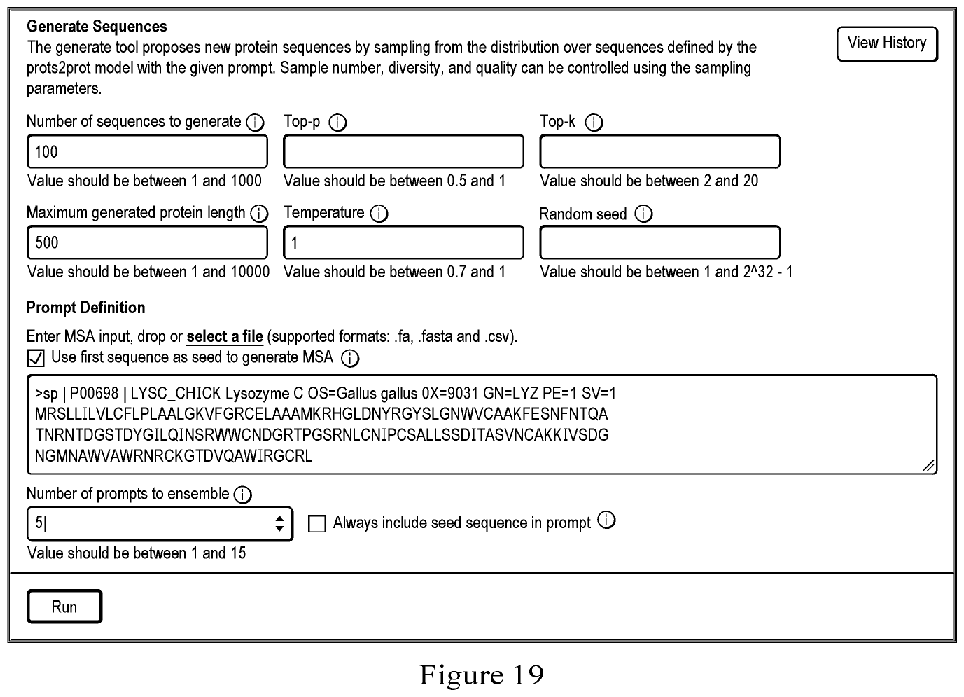

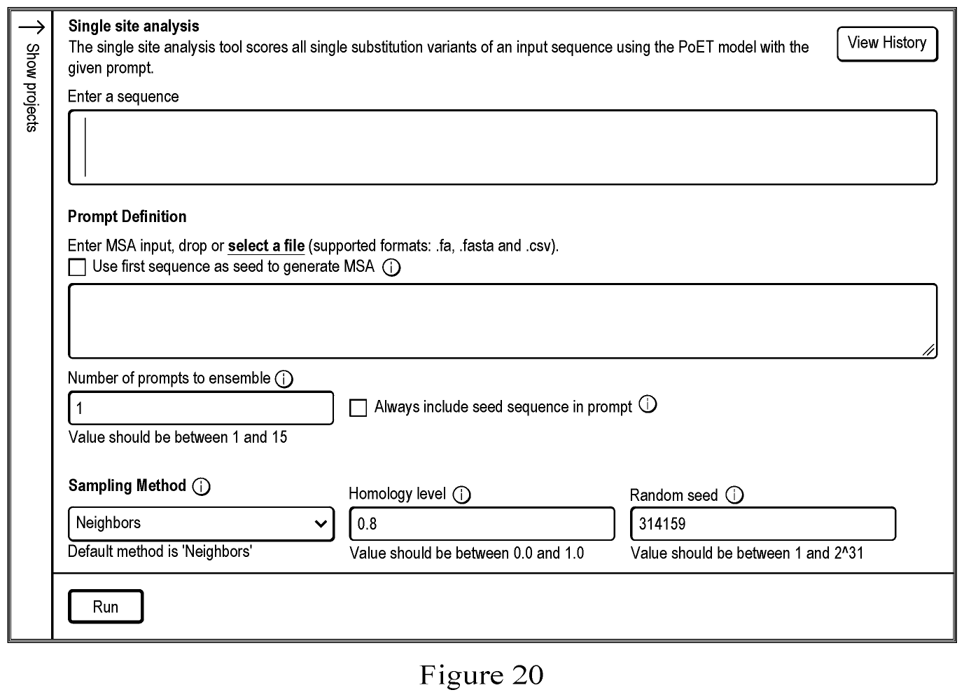

Representative interfaces for PoET prompting are depicted in FIGS. 17-20.

The nomenclature POET is not intended to be limiting.

Other language model variants (that use the order invariant transformer layer) may be implemented. Such model variants include, for example: simplifying the second attention module in the transformer layer by removing positional encoding; in an encoder-decoder framework, decoding only one sequence at a time instead of a sequence-of-sequences (but still conditioning on a sequence-of-sequences) by replacing the NxTieredTransformerDecoderLayer in component 604 (FIG. 6) with NxTransformerDecoderLayer; by dividing up the computation by using additional encoders and decoders; by tying the weights of any two transformer layers in a model; by adding additional sequence processing layers such as RNNs or SSMs, and so forth. An example of using additional encoders and decoders may be as follows: instead of using an encoder to embed a sequence-of-sequences and decoding each sequence in the sequence-of-sequences based on the embeddings of the individual sequences, an encoder may be used to embed each sequence-of-sequences without the sequence to decode; then, embed each sequence to decode conditioned on the former using cross attention, and then use these embeddings to decode each sequence.

### Enabling Technologies

Aspects of this disclosure may be practiced, typically in software, on one or more machines or computing devices. More generally, the techniques described herein are provided using a set of one or more computing-related entities (systems, machines, processes, programs, libraries, functions, or the like) that together facilitate or provide the described functionality described above. In a typical implementation, a representative machine on which the software executes comprises commodity hardware, an operating system, an application runtime environment, and a set of applications or processes and associated data, that provide the functionality of a given system or subsystem. As described, the functionality may be implemented in a standalone machine, or across a distributed set of machines. A computing device connects to the publicly-routable Internet, an intranet, a private network, or any combination thereof, depending on the desired implementation environment.

One implementation may be a machine learning-based computing platform. One or more functions of the computing platform may be implemented in a cloud-based architecture. The platform may comprise co-located hardware and software resources, or resources that are physically, logically, virtually and/or geographically distinct. Communication networks used to communicate to and from the platform services may be packet-based, non-packet based, and secure or non-secure, or some combination thereof.

Each above-described process or process step/operation preferably is implemented in computer software as a set of program instructions executable in one or more processors, as a special-purpose machine.

Representative machines on which the subject matter herein is provided may be hardware processor-based computers running an operating system and one or more applications to carry out the described functionality. One or more of the processes described above are implemented as computer programs, namely, as a set of computer instructions, for performing the functionality described. Virtual machines may also be utilized.

While the above describes a particular order of operations performed by certain embodiments of the invention, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

While the disclosed subject matter has been described in the context of a method or process, the subject matter also relates to apparatus for performing the operations herein. This apparatus may be a particular machine that is specially constructed for the required purposes, or it may comprise a computer otherwise selectively activated or reconfigured by a computer program stored in the computer. Such a computer program may be stored in a non-transitory computer readable storage medium, such as, but is not limited to, any type of disk including an optical disk, a CD-ROM, and a magnetic-optical disk, a read-only memory (ROM), a random access memory (RAM), a magnetic or optical card, or any type of media suitable for storing electronic instructions, and each coupled to a computer system bus.

There is no limitation on the type of computing entity that may implement a function or operation as described herein.

While given components of the system have been described separately, one of ordinary skill will appreciate that some of the functions may be combined or shared in given instructions, program sequences, code portions, and the like. Any application or functionality described herein may be implemented as native code, by providing hooks into another application, by facilitating use of the mechanism as a plug-in, by linking to the mechanism, and the like.

The functionality may be co-located or various parts/components may be separately and run as distinct functions, and in one or more locations over a distributed network.

Computing entities herein may be independent from one another, or associated with one another. Multiple computing entities may be associated with a single enterprise entity, but are separate and distinct from one another.

What is claimed here follows below:

1. A method, comprising:
training a language model to model a distribution over protein families, where each protein family is generated as a sequence-of-sequences, the language model comprising a transformer layer configured to capture order invariance between sequences while preserving order-dependence between tokens within sequences; and
performing a protein engineering workflow using the trained language model.

2. The method as described in claim 1 wherein the language model models the distribution P(X=x), where x=s₁, s₂, . . . , sₙ is a concatenation of n sequences sᵢ from a same family, and wherein each sequence sᵢ=s_{i,1}, s_{i,2}, . . . , s_{i,Li} is a sequence of Lᵢ amino acids padded by a start token, and an end token.

3. The method as described in claim 2, wherein the transformer layer comprises first and second attention modules.

4. The method as described in claim 3, wherein the first attention module is a within-sequence module in which a representation at each position of each sequence is updated based on attending only to other tokens within this sequence, and wherein the second attention module is a between-sequence module in which the representation at each position of each sequence is updated based on attending to all sequences within the sequence-of-sequences.

5. The method as described in claim 1, wherein the language model is trained using sets of homologous sequences.

6. The method as described in claim 1, wherein, following training, an order of sequences in a sequence-of-sequences is random.

7. The method as described in claim 1, wherein the protein engineering workflow is variant prioritization.

8. The method as described in claim 7, wherein variant prioritization assigns a score to each sequence in a set of variants {v₁, v₂, . . . , vₙ} of a target sequence t that accurately reflects a relative fitness of the variants, and predicts the fitness of a variant as a conditioned log-likelihood of the variant vᵢ given a set of sequences S homologous to the target t.

9. The method as described in claim 1, wherein the protein engineering workflow generates one or more protein sequences with a given function.

10. The method as described in claim 9, wherein the one or more protein sequences are generated by identifying a specific property of interest, and conditioning the model on only a subset of relevant homologs that are known to or are predicted to display the specific property of interest.

11. The method as described in claim 9, wherein the one or more protein sequences are generated by prompting the model with a known set of sequences, and then sampling from the model by using predicted next token probabilities to determine a sequence of amino acids.

12. The method as described in claim 9, wherein the one or more protein sequences are generated by prompting the model with a prompt that is augmented to include a natural language description.

13. The method as described in claim 12, wherein the prompt concatenates a natural language description of a protein, a sequence of the protein, and a natural language description of a target protein to be output from the model, wherein the target protein has a degree of similarity to the protein.

14. The method as described in claim 1, wherein the protein engineering workflow is sequence infilling.

15. The method as described in claim 14, sequence infilling comprises:
configuring a prompt with at least one region of a sequence masked with a masking token; and
applying the prompt to the model to generate the sequence, wherein the model replaces the masking token with one or more amino acid sequences.

16. The method as described in claim 1, wherein the protein engineering workflow is homology augmented learning.

17. The method as described in claim 16, wherein the homology augmented learning comprises mapping each of a set of protein sequences to a sequence of per-residue model embeddings, reducing each sequence of per-residue model embeddings to a fixed length vector to generate a reduced embedding, and training a supervised machine learning algorithm to learn a function mapping the reduced embeddings to measures of a given function.

18. The method as described in claim 1, wherein the protein engineering workflow is per-residue sequence annotation.

19. The method as described in claim 1, wherein the protein engineering workflow is 3D protein structure prediction.

20. The method as described in claim 19, wherein the 3D protein structure prediction uses a deep structure prediction model augmented to use per-residue embeddings generated by the language model.

21. The method as described in claim 1, wherein the language model comprises an encoder portion, and a decoder portion, the decoder portion having a cross-attention function that receives the output of the encoder portion.

22. The method as described in claim 21 wherein the language model implements one of: a prefix language modeling objective, a masked language modeling objective, and a combination of a prefix language model objective and a masked language modeling objective.

23. An apparatus, comprising:
a hardware processor; and
computer memory holding computer program code executed by the hardware processor, the computer program code configured to:
train a language model to model a distribution over protein families, where each protein family is generated as a sequence-of-sequences, the language model comprising a transformer layer configured to capture order invariance between sequences while preserving order-dependence between tokens within sequences; and
perform a protein engineering workflow using the trained language model.
